SEQUENCE LISTING (16 sequences)

SEQ ID NO: 1 - protein, 8 aa, synthetic construct
  source 1..8: note = Ova
SIINFEKL

SEQ ID NO: 2 - protein, 9 aa, synthetic construct
  source 1..9: note = DCT
SVYDFFVWL

SEQ ID NO: 3 - protein, 27 aa, synthetic construct
  source 1..27: note = B16Mut05
FVVKAYLPVNESFAFTADLRSNTGGQA

SEQ ID NO: 4 - protein, 27 aa, synthetic construct
  source 1..27: note = B16Mut17
VVDRNPQFLDPVLAYLMKGLCEKPLAS

SEQ ID NO: 5 - protein, 27 aa, synthetic construct
  source 1..27: note = B16Mut20
FRRKAFLHWYTGEAMDEMEFTEAESNM

SEQ ID NO: 6 - protein, 27 aa, synthetic construct
  source 1..27: note = B16Mut22
PKPDFSQLQRNILPSNPRVTRFHINWD

SEQ ID NO: 7 - protein, 27 aa, synthetic construct
  source 1..27: note = B16Mut25
STANYNTSHLNNDVWQIFENPVDWKEK

SEQ ID NO: 8 - protein, 27 aa, synthetic construct
  source 1..27: note = B16Mut28
NIEGIDKLTQLKKPFLVNNKINKIENI

SEQ ID NO: 9 - protein, 27 aa, synthetic construct
  source 1..27: note = B16Mut30
PSKPSFQEFVDWENVSPELNSTDQPFL

SEQ ID NO: 10 - protein, 27 aa, synthetic construct
  source 1..27: note = B16Mut44
EFKHIKAFDRTFANNPGPMVVFATPGM

SEQ ID NO: 11 - protein, 27 aa, synthetic construct
  source 1..27: note = B16Mut48
SHCHWNDLAVIPAGVVHNWDFEPRKVS

SEQ ID NO: 12 - protein, 27 aa, synthetic construct
  source 1..27: note = CT26Mut02
PLLPFYPPDEALEIGLELNSSALPPTE

SEQ ID NO: 13 - protein, 27 aa, synthetic construct
  source 1..27: note = CT26Mut03
DKPLRRNNSYTSYTMAICGMPLDSFRA

SEQ ID NO: 14 - protein, 27 aa, synthetic construct
  source 1..27: note = CT26Mut26
VILPQAPSGPSYAIYLQPAQAQMLTPP

SEQ ID NO: 15 - protein, 27 aa, synthetic construct
  source 1..27: note = CT26Mut27
EHIHRAGGLFVADAIQVGFGRIGKHFW

SEQ ID NO: 16 - protein, 27 aa, synthetic construct
  source 1..27: note = CT26Mut37
EVIQTSKYYMRDVIAIESAWLLELAPH

(19) United States
(12) Patent Application Publication
(10) Pub. No.: US 2023/0285552 A1
(43) Pub. Date: Sep. 14, 2023

(54) ONCOLYTIC VIRUSES AS ADJUVANTS

(71) Applicant: TURNSTONE LIMITED PARTNERSHIP, Toronto (CA)

(72) Inventors: Marie-Claude Bourgeois-Daigneault, Ottawa (CA); John Bell, Gatineau (CA)

(73) Assignee: TURNSTONE LIMITED PARTNERSHIP, Toronto, ON (CA)

(21) Appl. No.: 18/307,593

(22) Filed: Apr. 26, 2023

Related U.S. Application Data
(63) Continuation of application No. 16/971,285, filed on Aug. 19, 2020.
(60) Provisional application No. 62/751,091, filed on Oct. 26, 2018.
(60) Provisional application No. 62/633,883, filed on Feb. 22, 2018.

(51) Int. Cl. A61K 39/39; A61P 37/04; A61K 35/761; A61K 35/766; A61K 35/768; A61K 39/00

(52) U.S. Cl. CPC A61K 39/39; A61P 37/04; A61K 35/761; A61K 35/766; A61K 35/768; A61K 39/0011; A61K 2039/5252; A61K 2039/54; A61K 2039/545

(57) ABSTRACT

Herein is described oncolytic viruses for use as immunologic adjuvants. There is provided a method of adjuvanting an immune response to an antigenic protein in a mammalian subject by administering the oncolytic virus and at least one antigenic peptide, with the latter not encoded by the former. Without the requirement for the virus to encode the antigenic protein, therapies may be readily personalized or formulated. The virus may be attenuated or inactivated. Prime:boost therapies for tumours are also provided, in which the prime comprises at least one antigenic protein, the boost comprises a virus and at least one antigenic protein, the at least one antigenic protein of the prime and the at least one antigenic protein of the boost are based on the same at least one tumour associated antigen, and the at least one antigenic protein of the boost is not encoded by the virus of the boost.

### FIELD

The present disclosure relates to adjuvants for enhancing immune responses. More particularly, the disclosure relates to oncolytic viruses as adjuvants.

### BACKGROUND

Pathogens and disease cells comprise antigens that can be detected and targeted by the immune system, thus providing a basis for immune-base therapies, including immunogenic vaccines and immunotherapies. In the context of cancer treatment, for example, immunotherapy is predicated on the fact that cancer cells often have molecules on their cell surfaces that can be recognized and targeted.

Viruses have also been employed in cancer therapy, in part for their ability to directly kill disease cells. For example, oncolytic viruses (OVs) specifically infect, replicate in and kill malignant cells, leaving normal tissues unaffected. Several OVs have reached advanced stages of clinical evaluation for the treatment of various neoplasms (Russell S J. et al., (2012) Nat Biotechnol 30:658-670). In addition to the vesicular stomatitis virus (VSV) (Stojdl D F. et al., (2000) Nat Med 6:821-825; Stojdl D F. et al., (2003) Cancer Cell 4:263-275), other rhabdoviruses displaying oncolytic activity have been described recently (Brun J. et al., (2010) Mol Ther 18:1440-1449; Mahoney D J. et al., (2011) Cancer Cell 20:443-456). Among them, the non-VSV Maraba virus showed the broadest oncotropism in vitro (WO 2009/016433). A mutant Maraba virus with improved tumour selectivity and reduced virulence in normal cells was engineered. The attenuated strain is a double mutant strain containing both G protein (Q242R) and M protein (L123W) mutations. In vivo, this attenuated strain, called MG1 or Maraba MG1, demonstrated potent anti-tumour activity in xenograft and syngeneic tumour models in mice, with superior therapeutic efficacy than the attenuated VSV, VSVΔM51 (WO 2011/070440).

Various strategies have been developed to improve OV-induced anti-tumour immunity (Pol J. et al., (2012) Virus Adaptation and Treatment 4:1-21). The strategies take advantage of both the oncolytic activity of the OV itself, and the ability to generate immunity to tumour-associated antigens. One strategy, defined as an oncolytic vaccine, consists of expressing a tumour antigen from the OV (Russell S J. et al., (2012) Nat Biotechnol 30:658-670). Previously, it has been demonstrated that VSV could also be used as a cancer vaccine vector (Bridle B W. et al., (2010) Mol Ther 184:4269-4275). When applied in a heterologous prime:boost setting to treat a murine melanoma model, a VSV-human dopachrome tautomerase (hDCT) oncolytic vaccine not only induced an increased tumour-specific immunity to DCT but also a concomitant reduction in antiviral adaptive immunity. As a result, the therapeutic efficacy was dramatically improved with an increase of both median and long term survivals (WO 2010/105347). Three specific prime:boost combination therapies are disclosed in PCT Application No. PCT/CA2014/050118. The combination therapies include a lentivirus that encodes as an antigen: a Human Papilloma Virus (HPV) E6/E7 fusion protein, human Six-Transmembrane Epithelial Antigen of the Prostate (huSTEAP) protein, or Cancer Testis Antigen 1; and a Maraba MG1 virus that encodes the same antigen. PCT Application No. PCT/CA2014/050118 also discloses a prime:boost combination therapy using an adenovirus that encodes MAGEA3 as an antigen, and a Maraba MG1 virus that encodes the same antigen. PCT Application No. PCT/IB2017/000622 disclose combination prime:boost therapies involving oncolytic viruses that infect, replicate, and kill malignant cells. The viruses are engineered to encode and express antigenic proteins based on tumour associated antigens. The antigenic proteins (i) generate immunity and (ii) induce an immune response that yields a therapeutic effect.

It would be desirable to provide therapies that are more readily adaptable to targets, and/or susceptible to personalization.

### SUMMARY

The following summary is intended to introduce the reader to one or more inventions described herein but not to define any one of them.

It is an object of the present disclosure to obviate or mitigate at least one disadvantage of previous approaches.

It has surprisingly been found that oncolytic viruses can serve as adjuvants. The authors of the present disclosure have found that an oncolytic virus administered to a mammal can adjuvant an immune response to an administered antigenic protein that is not encoded by the virus. The therapies according to the present disclosure thus do not require a virus-encoded antigen. In the context of a prime:boost therapy, for example, the prime, the boost, or both may comprise a virus and a separate, non-virus-encoded antigenic protein. These results are unexpected, as it was previously thought that viral expression of an encoded antigen was important for stimulation of the immune response to the antigenic protein. With no need for modification of the oncolytic virus to encode the antigenic protein, therapies can be more adapted to different targets, e.g., using synthetic peptides. They can be more readily personalized, e.g., to target the tumour-associated antigens of a given tumour.

In one aspect, there is provided a combination prime:boost therapy for use in inducing an immune response in a mammalian subject, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one aspect, there is provided a composition comprising a prime or a boost for use in inducing an immune response in a mammalian subject in a combination prime:boost treatment, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one aspect, there is provided a composition comprising a prime for use in inducing an immune response in a mammalian subject in a combination prime:boost treatment, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one aspect, there is provided a composition comprising a boost for use in inducing an immune response in a mammalian subject in a combination prime:boost treatment, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In another aspect, there is provided a kit for use in inducing an immune response in a mammalian subject, wherein the kit comprises: a prime comprising at least one antigenic protein, formulated to generate the immune response in the mammal; and a boost comprising a virus and at least one antigenic protein, formulated to induce the immune response in the mammal; wherein the at least one antigenic protein of the prime and the at least one antigenic protein of the boost are based on the same at least one tumour associated antigen, wherein the at least one antigenic protein of the boost is not encoded by the virus of the boost.

In another aspect, there is provided a use of the combination prime:boost therapy described herein for treatment of a tumour in a mammalian subject.

In another aspect, there is provided the combination prime:boost therapy described herein for use in treatment of a tumour in a mammalian subject.

In another aspect, there is provided a method of treating a tumour in a mammalian subject, the method comprising administering to the subject the combination prime:boost therapy described herein.

In another aspect, there is provided a method for producing the combination prime:boost therapy described herein, the method comprising: synthesizing the at least one antigenic protein of the boost, and producing the combination prime:boost therapy.

In another aspect, there is provided a method for producing the combination prime:boost therapy described herein, the method comprising: synthesizing the at least one antigenic protein of the prime, and producing the combination prime:boost therapy.

In another aspect, there is provided a use of an oncolytic virus and at least one antigenic protein for inducing an immune response in a mammalian subject, wherein the at least one antigenic protein is not encoded by the oncolytic virus.

In another aspect, there is provided a use of an oncolytic virus for adjuvanting an immune response to at least one antigenic protein in a mammalian subject, wherein the at least one antigenic protein is not encoded by the oncolytic virus.

In another aspect, there is provided a method of adjuvanting an immune response to at least one antigenic protein in a mammalian subject, the method comprising administering to the subject an oncolytic virus and the at least one antigenic protein, wherein the at least one antigenic protein is not encoded by the virus.

In another aspect, there is provided an immunogenic composition comprising an oncolytic virus and at least one antigenic protein, wherein the at least one antigenic protein is not encoded by the oncolytic virus.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts a schematic representation of the treatment schedule used in experiments.

FIG. 2 shows IFNγ ELISPOT analysis of splenocytes harvested on day 21 from mice primed with adenovirus (Ad) expressing DCT peptide (termed ‘Ad-DCT’) and boosted with Maraba virus MG1 expressing DCT peptide (termed ‘MRB-DCT’, wherein is indicative of viral coding) or MRB co-administered with DCT peptide (termed ‘MRB+DCT’, where the ‘+’ is indicative of co-administration of a peptide that is not encoded by or part of the virus).

FIG. 3 shows that Ad-DCT alone induces an immune response to DCT (second group from the left), which is boosted by MRB+DCT to levels that are comparable to MRB-DCT (last group).

FIG. 4 shows flow cytometry analysis from the same experiment as in FIG. 3.

FIG. 5 shows IFNγ ELISPOT analysis of splenocytes harvested on day 21 from mice primed with Ad-DCT and boosted with MRB co-administered with DCT peptide using different routes—intravenous (IV), intratumoral (IT), or intramuscular (IM).

FIG. 6 shows IFNγ ELISPOT analysis of splenocytes harvested on day 21 from mice primed with Ad-DCT and boosted with either MRB or UV-inactivated MRB (UVMRB) co-administered with DCT peptide.

FIG. 7 shows IFNγ ELISPOT analysis of splenocytes harvested on day 21 from mice primed with Ad-DCT and boosted with either W, VSV or MV co-administered with DCT peptide.

FIG. 8 shows IFNγ ELISPOT analysis of splenocytes harvested on day 14 from mice primed with Ad-DCT or Ad or polyl:C co-administered with DCT peptide (all IM).

FIG. 9 shows IFNγ ELISPOT analysis of splenocytes harvested on day 21 from mice primed with Ad-DCT and boosted with MRB-DCT or MRB co-administered with DCT peptide.

FIG. 10 shows IFNγ ELISPOT analysis of splenocytes harvested on day 14 from mice primed with MRB, MRB-Ova or MRB co-administered with Ova peptide.

FIG. 11 shows IFNγ ELISPOT analysis of splenocytes harvested on day 21 from mice primed with Ad-Ova together or not with DCT peptide and boosted with MRB-Ova together or not with DCT peptide.

FIG. 12 depicts results for mice bearing established subcutaneous B16F10-Ova tumours treated IM with polyl:C and the indicated peptides on days 7 and 14.

FIG. 13 depicts results for mice bearing established subcutaneous CT26 tumours treated IM with polyl:C and the indicated peptide on days 7 and 14.

FIG. 14 shows IFNγ ELISPOT analysis of splenocytes harvested on day 14 from mice primed with polyl:C or MRB together with DCT peptide (SC and IV).

FIG. 15 shows IFNγ ELISPOT analysis of splenocytes harvested on day 14 from mice primed with polyl:C (SC) or MRB (IV) together with the indicated B16Mut peptide.

FIG. 16 shows that MRB can be used as an adjuvant for immune priming or boosting, but not both. It depicts the result of IFNγ ELISPOT analysis of splenocytes harvested on day 21 from mice primed with Ad-DCT or MRB together with DCT peptide and boosted with MRB co-administered with DCT peptide.

FIG. 17 shows that polyl:C induces stronger immune responses when administered together with peptide IM or SC. It depicts results of IFNγ ELISPOT analysis of splenocytes harvested on day 14 from mice primed with polyl:C co-administered with DCT peptide following different routes (IP, IV, IM or SC).

FIG. 18 depicts results of IFNγ ELISPOT analysis of splenocytes harvested on day 21 from mice primed with Ad-DCT or Ad co-administered with DCT peptide or Ad-Ova or Ad co-administered with Ova peptide (day 7) and boosted with MRB-DCT or MRB co-administered with DCT peptide or MRB-Ova or MRB co-administered with Ova peptide (day 14).

FIG. 19 depicts the survival analysis of mice primed with Ad or Ad co-administered with DCT peptide (day 7) and boosted with MRB or MRB co-administered with DCT peptide (day 14).

FIG. 20 depicts the survival analysis of mice primed with Ad or Ad together with mutanome peptides (B16Mut20, B16Mut30, B16Mut44 and B16Mut48) (day 7) and boosted with MRB or MRB co-administered with mutanome peptides (B16Mut20, B16Mut30, B16Mut44 and B16Mut48) (day 14).

FIG. 21 depicts the tumour growth analysis of mice primed with Ad or Ad together with mutanome peptides (CT26Mut20, CT26Mut27 and CT26Mut37) (day 7) and boosted with MRB or MRB co-administered with mutanome peptides (CT26Mut20, CT26Mut27 and CT26Mut37) (day 14).

FIG. 22 depicts survival analysis for the experiment in FIG. 21. It depicts the survival of mice primed with Ad or Ad together with mutanome peptides (CT26Mut20, CT26Mut27 and CT26Mut37) (day 7) and boosted with MRB or MRB co-administered with mutanome peptides (CT26Mut20, CT26Mut27 and CT26Mut37) (day 14).

FIG. 23 depicts the tumor growth analysis of mice primed with Ad-Ova or Ad-Ova together with mutanome peptides (B16Mut20, B16Mut30, B16Mut44 and B16Mut48) (day 7) and boosted with MRB-Ova or MRB-Ova co-administered with mutanome peptides (B16Mut20, B16Mut30, B16Mut44 and B16Mut48) (day 14).

FIG. 24 depicts survival analysis for the experiment in FIG. 23. It depicts the survival of mice primed with Ad-Ova or Ad-Ova together with mutanome peptides (B16Mut20, B16Mut30, B16Mut44 and B16Mut48) (day 7) and boosted with MRB-Ova or MRB-Ova co-administered with mutanome peptides (B16Mut20, B16Mut30, B16Mut44 and B16Mut48) (day 14).

### DETAILED DESCRIPTION

The present disclosure provides oncolytic viruses for use as immunologic adjuvants. Generally, the oncolytic viruses are capable of adjuvanting immune responses to antigenic proteins that are not encoded by the virus. In the context of prime:boost therapies, (1) the prime comprises an antigenic protein, and (2) the boost comprises a virus at and an antigenic protein, which is not encoded by the virus, with the antigenic protein of the prime and that of the boost based on the same antigen. The fact that the antigenic protein is not encoded by the virus means that the therapies may be readily adapted, may be readily personalized, or may be readily formulated.

Combination Prime:Boost Therapies for Cancer

In one aspect, there is provided a combination prime:boost therapy for use in inducing an immune response in a mammalian subject, wherein the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In the context of the present disclosure, a “combination prime:boost therapy” should be understood to refer to therapies for which (1) the at least one antigenic protein of the prime and (2) the virus and the at least one antigenic protein of the boost are to be administered as a prime:boost treatment. The prime and boost need not be physically provided or packaged together, since the prime is to be administered first and the boost is to be administered only after an immune response has been generated in the mammal. In some examples, the combination may be provided to a medical institute, such as a hospital or doctor's office, in the form of a package (or plurality of packages) of the prime, and a package (or plurality of packages) of the boost. The packages may be provided at different times. In other examples, the combination is provided to a medical institute, such as a hospital or doctor's office, in the form of a package that includes both the prime and the boost. A combination prime:boost therapy may also be referred as a combination prime:boost vaccine.

The term “mammal”, as used herein, refers to humans as well as non-human mammals. In one embodiment, the mammal may be a human.

By the term “antigenic protein” is meant any peptide comprising an immunogenic (antigenic) sequence that is capable of eliciting a biologically significant immune response.

By “tumour associated antigen” is meant any immunogen that is that is associated with tumour cells, and that is either absent from or less abundant in healthy cells or corresponding healthy cells (depending on the application and requirements). For instance, the tumour associated antigen may be unique, in the context of the organism, to the tumour cells.

By “not encoded by the virus”, as used herein, is mean that the at least one antigenic protein of the boost is not produced by transcription and translation of the nucleic acid sequences of the virus of the boost. The same applies when the term pertains to the prime in embodiments in which the prime comprises a virus and the at least one antigenic protein of the prime is not encoded by the virus of the prime. It will be understood that this does not preclude the virus, in each case, being modified or engineered. In certain embodiments, the antigenic proteins are not part of the viral particles. In certain embodiments, the antigenic proteins are not attached, conjugated, or otherwise physically connected to the viral particles. By this, is meant that there no covalent bonds between the antigenic proteins and the viral particles. In some embodiments, the antigenic proteins are not physically associated with the viral particles. Physically associated, in this context, indicates non-covalent interactions.

By “based on the same at least one tumour associated antigen” is meant that the at least one antigenic protein of the prime and the at least one antigenic protein of the boost are design or selected, such that they each comprise sequences eliciting an immune reaction to the same tumour associated antigen. It will be appreciated that the at least one antigenic protein of the prime and the at least one antigenic protein of the boost need not be exactly the same in order to accomplish this. For instance, they may be peptides comprising sequences that partially overlap, with the overlapping segment comprising a sequence corresponding to the tumour associated antigen, or a sequence designed to elicit an immune reaction to the tumour associated antigen, thereby allowing an effective prime and boost to the same antigen to be achieved. However, in one embodiment, the at least one antigenic protein of the prime and the at least one antigenic protein of the boost are the same.

In one embodiment, the at least one tumour associated antigen is based on the mutanome of a tumour of the mammalian subject.

By “mutanome” is meant the collective of an individual mammal's tumour-specific alterations and mutations, which encode a set of antigens that are specific to the subject. These are different from “shared” antigens, which are expressed in tumours from multiple patients and are typically normal, non-mutated self-proteins. Mutanome-encoded peptides may evoke a more vigorous T cell response due to a lack of thymic tolerance against them, and this immunity may be restricted to tumours, since the mutated gene product is only expressed in tumours (Overwijk et al.: Mining the mutanome: developing highly personalized Immunotherapies based on mutational analysis of tumours. Journal for ImmunoTherapy of Cancer 2013 1:11). The mutanome can be readily determined for a given tumour, e.g. by next generation sequencing.

In one embodiment, the at least one antigenic protein of the prime comprises a plurality antigenic proteins, and the at least one antigenic protein of the boost comprises a plurality of antigenic proteins, each of which is not encoded by the virus of the boost, and the plurality of antigenic proteins of the prime and the plurality of antigenic proteins of the boost are based on the same plurality of tumour associated antigens.

By “based on the same plurality of tumour associated antigens” it will be understood that, for each tumour associated antigen in the plurality, there will be at least one antigenic protein in the prime and at least one antigenic protein in the boost for that tumour associated antigen, such that each tumour associated antigen that is targeted will have at least one corresponding pair of prime/boost antigenic proteins. As above, it will be appreciated that plurality of antigenic proteins of the prime and the plurality of antigenic proteins of the boost need not be the same, and that pairs of antigenic proteins from the prime and boost may elicit an immune response to the same tumour associated antigen without being exactly the same. For instance, the pairs may be partially overlapping, with the overlapping segment comprising a sequence corresponding to the tumour associated antigen, or a sequence designed to elicit an immune response to the tumour associated antigen. However, in one embodiment, the plurality of antigenic proteins of the prime and the plurality of antigenic proteins of the boost are the same.

In one embodiment, the plurality of tumour associated antigens are based on the mutanome of a tumour the mammalian subject.

In one embodiment, the virus of the boost is an oncolytic virus.

By “oncolytic virus” is meant any one of a number of viruses that have been shown, when active, replicate and kill tumour cells in vitro or in vivo. These viruses may naturally oncolytic viruses, or virus that have been modified to produce or improve oncolytic activity. As used here, in certain embodiments the term may encompass attenuated, replication defective, inactivated, engineered, or otherwise modified forms of an oncolytic virus suited to purpose. Thus, in some embodiments it will be understood that what is termed an “oncolytic virus” for the purposes of description may not actually retain oncolytic activity. The use of inactive viruses can be desirable in context in which it is undesirable to administer active or “live” virus.

In one embodiment, the virus of the boost is a Rhabdovirus.

“Rhabdovirus” include, inter alia, one or more of the following viruses or variants thereof: Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus. In certain aspects, rhabdovirus can refer to the supergroup of Dimarhabdovirus (defined as rhabdovirus capable of infection both insect and mammalian cells).

In one embodiment, the Rhabdovirus is a Maraba virus or an engineered variant thereof.

By “engineered variant” will be understood a virus that has been genetically modified, e.g. by recombinant DNA technology. Such viruses may comprise, for example, mutations, insertions, deletions, or rearrangements relative to a wild-type virus.

In one embodiment, the virus of the boost is attenuated.

An “attenuated” virus is one having reduced the virulence, but which is still viable (or “live”).

In one embodiment, the virus of the boost is replication defective.

In one embodiment, the attenuated virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396, which is incorporated herein by reference, wherein it is referred to as SEQ ID NO: 4. An example of the Maraba G protein is described PCT Application No. PCT/IB2010/003396, wherein it is referred to as SEQ ID NO: 5. In one embodiment, the virus of the boost is the Maraba double mutant (“Maraba DM”) described in PCT Application No. PCT/IB2010/003396. In one embodiment, the virus of the boost is the “Maraba MG1” described in PCT Application No. PCT/CA2014/050118, which is incorporated herein by reference.

In one embodiment, the virus of the boost is an adenovirus, a vaccinia virus, measles virus, or a vesicular stomatitis virus.

In one embodiment, the virus of the boost is an adenovirus, a vaccinia virus, or a vesicular stomatitis virus.

In one embodiment, the boost is formulated for intravenous, intramuscular, or intratumoral administration.

In one embodiment, the prime is formulated for intravenous, intramuscular, or intratumoral administration.

In one embodiment, the virus of the boost is inactivated. In one embodiment, the virus of the boost is UV-inactivated.

In one embodiment, the prime additionally comprises a non-viral immunologic adjuvant.

By “immunologic adjuvant” will be understood a molecule that potentiates the immune response to an antigen and/or modulates it towards the desired immune response. One example is polyl:C.

In one embodiment, the prime additionally comprises a virus, wherein the virus of the prime is immunologically distinct from the virus of the boost.

By “immunologically distinct” will be understood that the viruses do not product antisera that cross react with one another. The use of immunological distinct viruses in the prime and boost permits an effective prime/boost response to the target antigen that is commonly targeted by the prime and boost. The virus of the prime may be any one of the above-described options for the virus of the boost, provided that the viruses of the prime and boost are immunologically distinct.

In one embodiment, the virus of the prime is an adenovirus. The virus of the prime may be tumour selective. For example, the adenovirus of the prime may comprise a deletion in E1 and E3, rendering the virus susceptible to p53 inactivation. Since many tumours lack p53, such a modification effective renders the virus tumour-specific, and hence oncolytic. In one embodiment, the adenovirus is of serotype 5.

The virus of the prime may encode the at least one antigenic protein of the prime. Where multiple antigenic proteins are used in the prime, some or all of them may be encoded by the virus of the prime. For example, the virus of the prime may comprise a plurality of virus types, each type being engineered to encode one of the antigenic proteins. However, in one embodiment, the at least one antigenic protein of the prime is/are not encoded by the virus of the prime. Where a plurality of antigenic proteins are used, in one embodiment none of them will be encoded by the virus of the prime.

In one embodiment, the virus of the prime may be attenuated. In one embodiment, wherein the virus of the prime is inactivated. In one embodiment, the virus of the prime is UV inactivated.

In one embodiment, the at least one antigenic protein of the prime comprises a synthetic peptide. In one embodiment, the synthetic peptide of the prime is a synthetic long peptide (SLP). The at least one antigenic protein of the prime may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

In one embodiment, the at least one antigenic protein of the boost comprises a synthetic peptide. In one embodiment, the synthetic peptide of the boost is a synthetic long peptide (SLP). The at least one antigenic protein of the boost may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

The combination prime:boost therapy may additionally include an immune-potentiating compound, such as cyclophosphamide (CPA), that increases the prime immune response to the tumour associated antigenic protein generated in the mammal by administrating the first virus. Cyclophosphamide is a chemotherapeutic agent that may lead to enhanced immune responses against the tumour associated antigenic protein.

Compositions for Use

In one aspect, there is provided a composition comprising a prime or a boost for use in inducing an immune response in a mammalian subject in a combination prime:boost treatment, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one aspect, there is provided a composition comprising a prime for use in inducing an immune response in a mammalian subject in a combination prime:boost treatment, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one aspect, there is provided a composition comprising a boost for use in inducing an immune response in a mammalian subject in a combination prime:boost treatment, wherein: the prime comprises at least one antigenic protein, formulated to generate the immune response in the mammal; and the boost comprises a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one embodiment, the at least one antigenic protein of the prime and the at least one antigenic protein of the boost are the same.

In one embodiment, the at least one tumour associated antigen is based on the mutanome of a tumour of the mammalian subject.

In one embodiment, the at least one antigenic protein of the prime comprises a plurality antigenic proteins, and the at least one antigenic protein of the boost comprises a plurality of antigenic proteins, each of which is not encoded by the virus of the boost, and the plurality of antigenic proteins of the prime and the plurality of antigenic proteins of the boost are based on the same plurality of tumour associated antigens.

In one embodiment, the plurality of antigenic proteins of the prime and the plurality of antigenic proteins of the boost are the same.

In one embodiment, the plurality of tumour associated antigens are based on the mutanome of a tumour the mammalian subject.

In one embodiment, the virus of the boost is an oncolytic virus.

In one embodiment, the virus of the boost is a Rhabdovirus.

In one embodiment, the Rhabdovirus is a Maraba virus or an engineered variant thereof.

In one embodiment, the virus of the boost is attenuated.

In one embodiment, the virus of the boost is replication defective.

In one embodiment, the attenuated virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396, which is incorporated herein by reference, wherein it is referred to as SEQ ID NO: 4. An example of the Maraba G protein is described PCT Application No. PCT/IB2010/003396, wherein it is referred to as SEQ ID NO: 5. In one embodiment, the virus of the boost is the Maraba double mutant (“Maraba DM”) described in PCT Application No. PCT/IB2010/003396. In one embodiment, the virus of the boost is the “Maraba MG1” described in PCT Application No. PCT/CA2014/050118, which is incorporated herein by reference.

In one embodiment, the virus of the boost is an adenovirus, a vaccinia virus, measles virus, or a vesicular stomatitis virus.

In one embodiment, the virus of the boost is an adenovirus, a vaccinia virus, or a vesicular stomatitis virus.

In one embodiment, the boost is formulated for intravenous, intramuscular, or intratumoral administration.

In one embodiment, the prime is formulated for intravenous, intramuscular, or intratumoral administration.

In one embodiment, the virus of the boost is inactivated. In one embodiment, the virus of the boost is UV-inactivated.

In one embodiment, the prime additionally comprises a non-viral immunologic adjuvant. One example is polyl:C.

In one embodiment, the prime additionally comprises a virus, wherein the virus of the prime is immunologically distinct from the virus of the boost.

In one embodiment, the virus of the prime is an adenovirus. The virus of the prime may be tumour selective. For example, the adenovirus of the prime may comprise a deletion in E1 and E3, rendering the virus susceptible to p53 inactivation. Since many tumours lack p53, such a modification effective renders the virus tumour-specific, and hence oncolytic. In one embodiment, the adenovirus is of serotype 5.

The virus of the prime may encode the at least one antigenic protein of the prime. Where multiple antigenic proteins are used in the prime, some or all of them may be encoded by the virus of the prime. For example, the virus of the prime may comprise a plurality of virus types, each type being engineered to encode one of the antigenic proteins. However, in one embodiment, the at least one antigenic protein of the prime is/are not encoded by the virus of the prime. Where a plurality of antigenic proteins are used, in one embodiment none of them will be encoded by the virus of the prime.

In one embodiment, the virus of the prime may be attenuated. In one embodiment, wherein the virus of the prime is inactivated. In one embodiment, the virus of the prime is UV inactivated.

In one embodiment, the at least one antigenic protein of the prime comprises a synthetic peptide. In one embodiment, the synthetic peptide of the prime is a synthetic long peptide (SLP). The at least one antigenic protein of the prime may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

In one embodiment, the at least one antigenic protein of the boost comprises a synthetic peptide. In one embodiment, the synthetic peptide of the boost is a synthetic long peptide (SLP). The at least one antigenic protein of the boost may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

The composition for use may additionally include an immune-potentiating compound, such as cyclophosphamide (CPA), that increases the prime immune response to the tumour associated antigenic protein generated in the mammal by administrating the first virus. Cyclophosphamide is a chemotherapeutic agent that may lead to enhanced immune responses against the tumour associated antigenic protein.

In certain embodiments, the antigenic proteins are not attached, conjugated, or otherwise physically connected to the viral particles. In some embodiments, the antigenic proteins are not physically associated with the viral particles.

Kits for Inducing an Immune Response to a Tumour

In one aspect, there is provide a kit for use in inducing an immune response in a mammalian subject, wherein the kit comprises a prime comprising at least one antigenic protein, formulated to generate the immune response in the mammal; and a boost comprising a virus and at least one antigenic protein, formulated to induce the immune response in the mammal; wherein the at least one antigenic protein of the prime and the at least one antigenic protein of the boost are based on the same at least one tumour associated antigen, and wherein the at least one antigenic protein of the boost is not encoded by the virus of the boost.

In one embodiment, the mammal may be a human.

In one embodiment, the at least one antigenic protein of the prime and the at least one antigenic protein of the boost are the same.

In one embodiment, the at least one tumour associated antigen is based on the mutanome of a tumour of the mammalian subject.

In one embodiment, the at least one antigenic protein of the prime comprises a plurality antigenic proteins, and the at least one antigenic protein of the boost comprises a plurality of antigenic proteins, each of which is not encoded by the virus of the boost, and the plurality of antigenic proteins of the prime and the plurality of antigenic proteins of the boost are based on the same plurality of tumour associated antigens. As above, it will be appreciated that plurality of antigenic proteins of the prime and the plurality of antigenic proteins of the boost need not be the same, and that pairs of antigenic proteins from the prime and boost may elicit an immune response to the same tumour associated antigen without being the same. For instance, the pairs may be partially overlapping, with the overlapping segment comprising a sequence corresponding to the tumour associated antigen, or a sequence designed to elicit an immune response to the tumour associated antigen. However, in one embodiment, the plurality of antigenic proteins of the prime and the plurality of antigenic proteins of the boost are the same.

In one embodiment, the plurality of tumour associated antigens are based on the mutanome of a tumour the mammalian subject.

In one embodiment, the virus of the boost is an oncolytic virus.

In one embodiment, the virus of the boost is a Rhabdovirus. The Rhabdovirus may be any of those listed above.

In one embodiment, the Rhabdovirus is a Maraba virus or an engineered variant thereof.

In one embodiment, the virus of the boost is an attenuated virus.

In one embodiment, the attenuated virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396, wherein it is referred to as SEQ ID NO: 4. An example of the Maraba G protein is described PCT Application No. PCT/IB2010/003396, wherein it is referred to as SEQ ID NO: 5. In one embodiment, the virus of the boost is the Maraba double mutant (“Maraba DM”) described in PCT Application No. PCT/IB2010/003396. In one embodiment, the virus of the boost is the “Maraba MG1” described in PCT Application No. PCT/CA2014/050118.

In one embodiment, the virus of the boost is an adenovirus, a vaccinia virus, measles virus, or a vesicular stomatitis virus.

In one embodiment, the virus of the boost is an adenovirus, a vaccinia virus, or a vesicular stomatitis virus.

In one embodiment, the boost is formulated for intravenous, intramuscular, or intratumoral administration.

In one embodiment, the prime is formulated for intravenous, intramuscular, or intratumoral administration.

In one embodiment, the virus of the boost is inactivated. In one embodiment, the virus of the boost is UV-inactivated.

In one embodiment, the prime additionally comprises a non-viral adjuvant.

In one embodiment, the prime additionally comprises a virus, wherein the virus of the prime is immunologically distinct from the virus of the boost.

In one embodiment, the virus of the prime is an adenovirus. The virus of the prime may be tumour selective. For example, the adenovirus of the prime may comprise a deletion in E1 and E3, rendering the virus susceptible to p53 inactivation. Since many tumours lack p53, such a modification effective renders the virus tumour-specific, and hence oncolytic.

The virus of the prime may encode the at least one antigenic protein of the prime. Where multiple antigenic proteins are used in the prime, some or all of them may be encoded by the virus of the prime. For example, the virus of the prime may comprise a plurality of virus types, each type being engineered to encode one of the antigenic proteins. However, in one embodiment, the at least one antigenic protein of the prime is/are not encoded by the virus of the prime. Where a plurality of antigenic proteins are used, in one embodiment none of them will be encoded by the virus of the prime.

In one embodiment, the virus of the prime may be attenuated. In one embodiment, wherein the virus of the prime is inactivated. In one embodiment, the virus of the prime is UV inactivated.

In one embodiment, the at least one antigenic protein of the prime comprises a synthetic peptide. In one embodiment, the synthetic peptide of the prime is a synthetic long peptide. The at least one antigenic protein of the prime may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

In one embodiment, the at least one antigenic protein of the boost comprises a synthetic peptide. In one embodiment, the synthetic peptide of the boost is a synthetic long peptide. The at least one antigenic protein of the prime may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

The kit may additionally include an immune-potentiating compound, such as cyclophosphamide (CPA), that increases the prime immune response to the tumour associated antigenic protein generated in the mammal by administrating the first virus. Cyclophosphamide is a chemotherapeutic agent that may lead to enhanced immune responses against the tumour associated antigenic protein.

In certain embodiments, the antigenic proteins are not attached, conjugated, or otherwise physically connected to the viral particles. In some embodiments, the antigenic proteins are not physically associated with the viral particles.

Therapeutic Prime:Boost Uses and Methods for Cancer

In one aspect, there is provided a use of the combination prime:boost therapy herein described for treatment of a tumour in a mammalian subject.

In one aspect, there is provided a combination prime:boost therapy herein described for use in treatment of a tumour in a mammalian subject.

In one aspect, there is provided a method of treating a tumour in a mammalian subject, the method comprising administering to the subject the combination prime:boost herein described.

In one aspect, there is provided a use of the composition for use, as defined above, for treatment of a tumour in a mammalian subject.

In one aspect, there is provided the composition for use, as defined above, in treatment of a tumour in a mammalian subject.

Production Methods

In one aspect, there is provided a method for producing the combination prime:boost therapy herein described, the method comprising synthesizing the at least one antigenic protein of the boost, and producing the combination prime:boost therapy.

In one aspect, there is provided a method for producing the combination prime:boost therapy herein described, the method comprising synthesizing the at least one antigenic protein of the prime, and producing the combination prime:boost therapy.

In one embodiment, the step of synthesizing comprises long peptide synthesis.

In one embodiment, the method further comprising, prior to the step of synthesizing, selecting the at least one tumour associated antigen based on the mutanome of the tumour of the mammalian subject.

In one embodiment, the method may also comprise determining the mutanome for a subject to determine unique peptides. Once determined, some embodiments include selecting target peptides from the mutanome. Some embodiments comprise predicting optimal targets, e.g. based on predicted antigenicity.

Uses for Adjuvanting an Immune Response

In one aspect, there is provided a use of an oncolytic virus and at least one antigenic protein for inducing an immune response in a mammalian subject, wherein the at least one antigenic protein is not encoded by the oncolytic virus.

In one aspect, there is provided an oncolytic virus and at least one antigenic protein for use in inducing an immune response in a mammalian subject, wherein the at least one antigenic protein is not encoded by the oncolytic virus.

In one aspect, there is provided a use of an oncolytic virus for adjuvanting an immune response to at least one antigenic protein in a mammalian subject, wherein the at least one antigenic protein is not encoded by the oncolytic virus.

In one aspect, there is provided an oncolytic virus for use in adjuvanting an immune response to at least one antigenic protein in a mammalian subject, wherein the at least one antigenic protein is not encoded by the oncolytic virus.

In one embodiment, the mammal is a human.

In one embodiment, the immune response is a therapeutic immune response.

In one embodiment, the mammalian subject has pre-existing immunity to the at least one antigenic protein.

“Pre-existing immunity” will be understood as a subject who is not naïve to a particular antigen, having previously been exposed to it. This may arise, for example, due to priming the subject with the antigen. It may also arise because the subject has low-level immunity because the antigen is present in the subject. For example, in the context of cancer, a subject may have low level prior immunity because of a tumour associated antigen is expressed by the tumour.

In one embodiment, the at least one antigenic protein is based on at least one tumour associated antigen.

In one embodiment, the at least one tumour associated antigen is based on the mutanome of a tumour the mammalian subject.

In one embodiment, the at least one antigenic protein comprises a plurality antigenic proteins.

In one embodiment, the plurality of antigenic proteins are based on the mutanome of a tumour the mammalian subject.

In one embodiment, the oncolytic virus is a Rhabdovirus. The Rhabdovirus may be any of those listed above.

In one embodiment, the Rhabdovirus is a Maraba virus or an engineered variant thereof.

In one embodiment, the oncolytic virus is an attenuated virus.

In one embodiment, the attenuated virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396, wherein it is referred to as SEQ ID NO: 4. An example of the Maraba G protein is described PCT Application No. PCT/IB2010/003396, wherein it is referred to as SEQ ID NO: 5. In one embodiment, the virus of the boost is the Maraba double mutant (“Maraba DM”) described in PCT Application No. PCT/IB2010/003396. In one embodiment, the virus of the boost is the “Maraba MG1” described in PCT Application No. PCT/CA2014/050118.

In one embodiment, the virus is an adenovirus, a vaccinia virus, measles virus, or a vesicular stomatitis virus.

In one embodiment, the virus of the boost is an adenovirus, a vaccinia virus, or a vesicular stomatitis virus.

In one embodiment, the virus and the at least one antigenic protein are formulated for intravenous, intramuscular, or intratumoral administration.

In one embodiment, the virus is inactivated.

In one embodiment, virus is UV-inactivated.

In one embodiment, the at least one antigenic protein comprises a synthetic peptide. In one embodiment, synthetic peptide comprises a long synthetic peptide. The at least one antigenic protein may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

Methods of Adjuvanting

In one aspect, there is provided a method of adjuvanting an immune response to at least one antigenic protein in a mammalian subject, the method comprising administering to the subject an oncolytic virus and the at least one antigenic protein, wherein the at least one antigenic protein is not encoded by the oncolytic virus.

In one embodiment, the mammal is a human.

In one embodiment, the step of administering comprises co-administering.

In one embodiment, the immune response is a therapeutic immune response.

In one embodiment, the mammalian subject has pre-existing immunity to the at least one antigenic protein.

In one embodiment, the at least one antigenic protein is based on at least one tumour associated antigen.

In one embodiment, the at least one tumour associated antigen is based on the mutanome of a tumour the mammalian subject.

In one embodiment, the at least one antigenic protein comprises a plurality antigenic proteins.

In one embodiment, the plurality of antigenic proteins are based on the mutanome of a tumour the mammalian subject.

In one embodiment, the oncolytic virus is a Rhabdovirus. The Rhabdovirus may be any of those listed above.

In one embodiment, the Rhabdovirus is a Maraba virus or an engineered variant thereof.

In one embodiment, the virus is an attenuated virus.

In one embodiment, the attenuated virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396, wherein it is referred to as SEQ ID NO: 4. An example of the Maraba G protein is described PCT Application No. PCT/IB2010/003396, wherein it is referred to as SEQ ID NO: 5. In one embodiment, the virus of the boost is the Maraba double mutant (“Maraba DM”) described in PCT Application No. PCT/IB2010/003396. In one embodiment, the virus of the boost is the “Maraba MG1” described in PCT Application No. PCT/CA2014/050118.

In one embodiment, the virus is an adenovirus, a vaccinia virus, measles virus, or a vesicular stomatitis virus.

In one embodiment, the virus of the boost is an adenovirus, a vaccinia virus, or a vesicular stomatitis virus.

In one embodiment, the step of administering is intravenous, intramuscular, or intratumoral.

In one embodiment, the virus is inactivated.

In one embodiment, the virus is UV-inactivated.

In one embodiment, the at least one antigenic protein comprises a synthetic peptide. In one embodiment, the synthetic peptide comprises a long synthetic peptide. The at least one antigenic protein may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

Immunogenic Compositions

In one aspect, there is provided an immunogenic composition comprising an oncolytic virus and at least one antigenic protein, wherein the at least one antigenic protein is not encoded by the oncolytic virus.

In one embodiment, the at least one antigenic protein is based on at least one tumour associated antigen.

In one embodiment, the at least one tumour associated antigen is based on the mutanome of a tumour a mammalian subject.

In one embodiment, the at least one antigenic protein comprises a plurality antigenic proteins.

In one embodiment, the plurality of antigenic proteins are based on the mutanome of a tumour the mammalian subject.

In one embodiment, the oncolytic virus is a Rhabdovirus. The Rhabdovirus may be any of those listed above.

In one embodiment, the Rhabdovirus is a Maraba virus or an engineered variant thereof.

In one embodiment, the virus is an attenuated virus.

In one embodiment, the attenuated virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/IB2010/003396, wherein it is referred to as SEQ ID NO: 4. An example of the Maraba G protein is described PCT Application No. PCT/IB2010/003396, wherein it is referred to as SEQ ID NO: 5. In one embodiment, the virus of the boost is the Maraba double mutant (“Maraba DM”) described in PCT Application No. PCT/IB2010/003396. In one embodiment, the virus of the boost is the “Maraba MG1” described in PCT Application No. PCT/CA2014/050118.

In one embodiment, the virus is an adenovirus, a vaccinia virus, measles virus, or a vesicular stomatitis virus.

In one embodiment, the virus of the boost is an adenovirus, a vaccinia virus, or a vesicular stomatitis virus.

In one embodiment, the virus is inactivated.

In one embodiment, the virus is UV-inactivated.

In one embodiment, the at least one antigenic protein comprises a synthetic peptide. In one embodiment, the synthetic peptide comprises a long synthetic peptide. The at least one antigenic protein may be 8 to 250 amino acids in length. Within this range, it may at least 10, at least 20, at least 30, at least 40, or at least 50 amino acids in length. With all these applicable ranges, may be less than 200, less than 150, less than 125, less than 100, less than 75, less than 50, less than 40, or less than 30 amino acids in length. Any combination of the stated upper and lower limits is envisaged.

### EXAMPLES

Materials and Methods

Cell Lines and Culture

B16F10 stably expressing ovalbumin were obtained from Dr. Rebecca Auer. Vero, HEK 293T and HeLa cells were all obtained from the American Type Culture Collection (ATCC). The cell lines were maintained in Dulbecco's Modified Eagle's Medium (DMEM) (Corning Cellgro) supplemented with 10% fetal bovine serum (FBS) (Sigma Life Science) and cultured at 37° C. with 5% CO2.

Viruses, Production and Quantification

The Maraba (MRB) virus used in this study is the clinical candidate variant MG1, such that in the ensuing text reference to ‘MRB’ should be understood as meaning MG1. The Vesicular stomatitis virus (VSV) used in this study is the mutant Δ51. Production and purification of VSV and MRB: Vero cells were infected at a multiplicity of infection (MOI) of 0.01 for 24 hours before harvesting, filtration [0.22 μm bottle top filter (Millipore)], and centrifugation (90 minutes at 30100 g) of the culture supernatant. The pellet was resuspended in Dulbecco's phosphate buffered saline (DPBS) (Corning Cellgro) and stored at −80° C. Viral titers were determined by plaque assay. Briefly, serially diluted samples were transferred to monolayers of Vero cells, incubated for 1 hour, and then overlaid with 0.5% agarose/DMEM supplemented with 10% FBS. Plaques were counted 24 hours later.

The Adenoviruses (Ad) used in this study (Ad, Ad-Ova and Ad-DCT) were all obtained from B. Lichty (all serotype E5). Production and purification of Ad: HEK 293T cells were infected at an MOI of 1 for 48 h in DMEM supplemented with 2% FBS. The infected cells were then collected and the pellet was frozen and thawed for 3 cycles. The debris were then removed by centrifugation and the cleared supernatant was centrifugated on a cesium chloride gradient (1.4 g/cm3 CsCl-1.2 g/cm3 CsCl) at 28000 rmp for 3.5 h at 4° C. The band corresponding to the Ad particles was then extracted and the virus was stored at −20° C. Viral titers were obtained using the Adeno-X rapid titer kit according to the manufacturer's protocol (Takara).

The vaccinia virus (VV) used in this study is the wild type Copenhagen strain.

The measles virus (MV) used in this study (Schwarts strain) expressed GFP and was a generous gift from Dr. Guy Ungerechts.

Irradiated Virus Maraba was UV-inactivated by exposure to 120 mJ/cm2 for 2 minutes using a Spectrolinker XL-1000 UV crosslinker as described previously (35).

Flow Cytometry

Spleens were harvested and mashed through a 70 μm strainer (Fisher Scientific) before lysis of red blood cells using ACK lysis buffer and resuspension in FACS buffer (PBS, 3% FBS). The splenocytes were re-stimulated ex-vivo using 2 μg/mL of the corresponding peptide and golgi-plug (BD Bioscience) was added to the mixture after 1 h for an additional 5 h in order to prevent cytokine secretion. Cells were stained using CD45, CD3, CD8, TNFα and IFNγ antibodies (all from BD Bioscience). The intracellular stainings were performed upon fixation and permeabilization of the cells (using the intracellular fixation and permeabilization buffer set (eBioscience)). Flow cytometry analysis was performed on a LSR Fortessa flow cytometer (BD biosciences).

Peptides

All peptides were obtained from Biomer Technology and have the amino acid sequences shown in Table 1.

TABLE 1
Peptide Sequences

| Peptide | Amino Acid Sequence |
|---|---|
| Ova | SIINFEKL |
| DCT | SVYDFFVWL |
| B16Mut05 | FVVKAYLPVNESFAFTADLRSNTGGQA |
| B16Mut17 | VVDRNPQFLDPVLAYLMKGLCEKPLAS |
| B16Mut20 | FRRKAFLHWYTGEAMDEMEFTEAESNM |
| B16Mut22 | PKPDFSQLQRNILPSNPRVTRFHINWD |
| B16Mut25 | STANYNTSHLNNDVWQIFENPVDWKEK |
| B16Mut28 | NIEGIDKLTQLKKPFLVNNKINKIENI |
| B16Mut30 | PSKPSFQEFVDWENVSPELNSTDQPFL |
| B16Mut44 | EFKHIKAFDRTFANNPGPMVVFATPGM |
| B16Mut48 | SHCHWNDLAVIPAGVVHNWDFEPRKVS |
| CT26Mut02 | PLLPFYPPDEALEIGLELNSSALPPTE |
| CT26Mut03 | DKPLRRNNSYTSYTMAICGMPLDSFRA |
| CT26Mut26 | VILPQAPSGPSYAIYLQPAQAQMLTPP |
| CT26Mut27 | EHIHRAGGLFVADAIQVGFGRIGKHFW |
| CT26Mut37 | EVIQTSKYYMRDVIAIESAWLLELAPH |

ELISPOT

Mouse IFNγ ELISPOTs (MabTech) were performed according to the manufacturer's protocol using splenocytes extracted 7 days after the last immunization. The incubation was performed for 24 h in serum-free DMEM using 2 μg/mL of peptide for re-stimulation.

In Vivo Experiments and Tumour Models

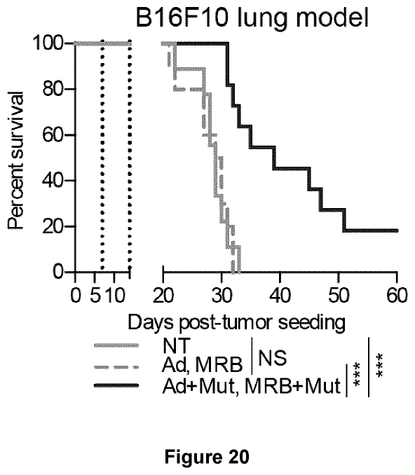

All experiments were performed in accordance with the University of Ottawa ACVS guidelines. Subcutaneous tumour model: 10⁶ or 10⁵ B16F10-Ova cells were injected into the left flank or IV of 6-8 weeks old female C57/B16 mice for the SC and the lung cancer model, respectively (Charles River Laboratories). For the CT26 SC tumour model, 10⁶ cells were injected into the left flank of Balb/c mice (Charles River Laboratories). Ad (1×10⁸ PFU) was administered intramuscularly in the quadriceps and MRB, VSV, MV and VV (all at a dose of 1×10⁸ PFU) were administered intravenously (unless specified otherwise) via the tail vein of the mice. Polyl:C was purchased from Invivogen and a dose of 50 ug was used per animal per immunization. The peptides (100 ug/mouse/immunization) were pre-mixed with the different viruses or with polyl:C prior to injection in a total volume of 100 uL. Immune priming and boosting were performed 7 and 14 days post-tumour seeding and the immune analysis was performed 7 days after the last immunization. For the experiment using several peptides (FIGS. 20 to 24), a total dose of 100 ug of peptide was used per immunization. For the efficacy experiments, the tumours were measured over time using electronic calipers.

Results and Discussion

In these experiments MRB indicates MG1. All experiments were done is tumour-bearing animals. The prime is to be understood as immunization at day 7, and the boost took place at day 14.

The results indicate that it is not necessary for the antigenic protein to be encoded by the virus to stimulate an immune response.

The viruses can be used as an adjuvant for immune boosting.

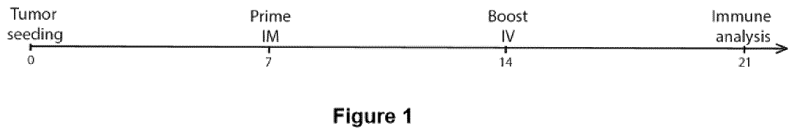

FIG. 1 shows a schematic representation of the treatment schedule used in this study.

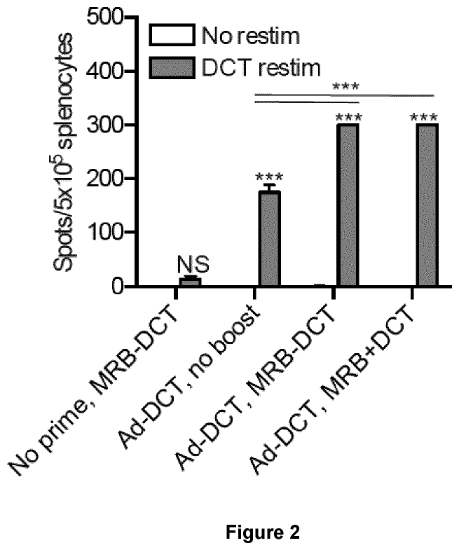

FIG. 2 shows IFNγ ELISPOT analysis of splenocytes harvested on day 21 from mice primed with adenovirus (Ad) expressing DCT peptide (termed ‘Ad-DCT’) and boosted with Maraba virus MG1 expressing DCT peptide (termed ‘MRB-DCT’) or MRB co-administered with DCT peptide (termed ‘MRB+DCT’, where the ‘+’ is indicative of co-administration of peptide not encoded by or part of the virus).

The results of FIG. 2 show that Ad-DCT alone induces an immune response to DCT (second group from the left). Immune boosting using MRB+DCT (last group on right) improves this immune response to levels that are comparable to MRB-DCT (third group from the left). FIG. 2 thus shows that MRB+DCT is as good as MRB-DCT as a boost in the heterologous virus prime-boost setting. There is no need for an MRB-encoded antigenic peptide. Unless indicated otherwise, the stats refer to the comparison between the “No restim” and “DCT restim” conditions. NS: p>0.05, ***: p<0.001 (unpaired multiple two-tailed t-test).

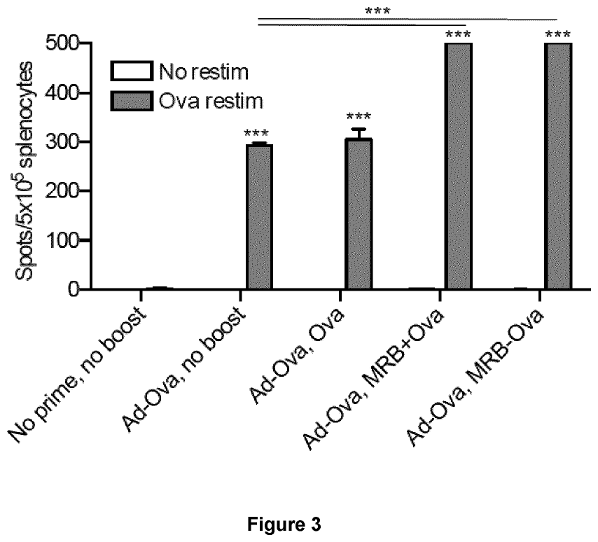

FIG. 3 shows IFNγ ELISPOT analysis of splenocytes harvested on day 21 from mice primed with Ad-Ova and boosted with MRB-Ova or MRB co-administered with Ova peptide (MRB+Ova). The results show that Ad-Ova alone induces an immune response to Ova (second group from the left). This immune response could not be boosted by the IV injection of Ova peptide alone (third group from the left). Immune boosting using MRB-Ova improves this immune response (last group) to levels that are comparable to MRB co-administered with Ova peptide (fourth group from the left). FIG. 3 shows that MRB+Ova is as good as MRB-Ova as a boost in the heterologous virus prime-boost setting, confirming the above result for DCT in FIG. 2. Unless indicated otherwise, the stats refer to the comparison between the “No restim” and “Ova restim” conditions. NS: p>0.05, ***: p<0.001 (unpaired multiple two-tailed t-test).

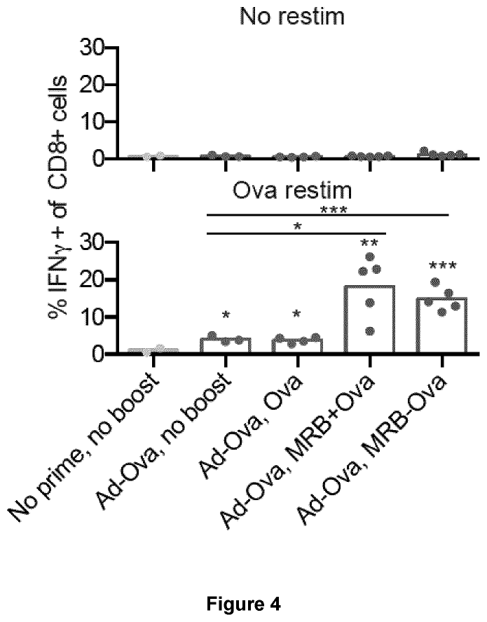

FIG. 4 shows flow cytometry analysis from the same experiment as in FIG. 3. Once again, the results show that Ad-Ova alone induces an immune response to Ova (second group from the left). This immune response could not be boosted by the IV injection of Ova peptide alone (third group from the left). Immune boosting using MRB-Ova improves this immune response (last group) to levels that are comparable to MRB co-administered with Ova (fourth group from the left). FIG. 4 thus confirms that MRB+Ova is as good as MRB-Ova as a boost in the heterologous virus prime-boost setting (again confirming the results seen for DCT in the context of another peptide). Unless indicated otherwise, the stats refer to the comparison between the “No restim” and “Ova restim” conditions. NS: p>0.05, *: p<0.05, **: p<0.01, ***: p<0.001 (unpaired multiple two-tailed t-test).

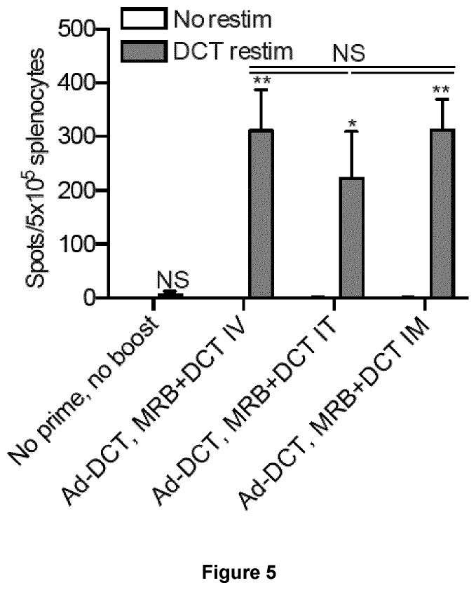

FIG. 5 shows IFNγ ELISPOT analysis of splenocytes harvested on day 21 from mice primed with Ad-DCT and boosted with MRB co-administered with DCT peptide using different routes (IV, IT or IM). The results show that all routes of administration of MRB+peptide induce comparable immune responses. Unless indicated otherwise, the stats refer to the comparison between the “No restim” and “DCT restim” conditions. NS: p>0.05, *: p<0.05, **: p<0.01 (unpaired multiple two-tailed t-test).

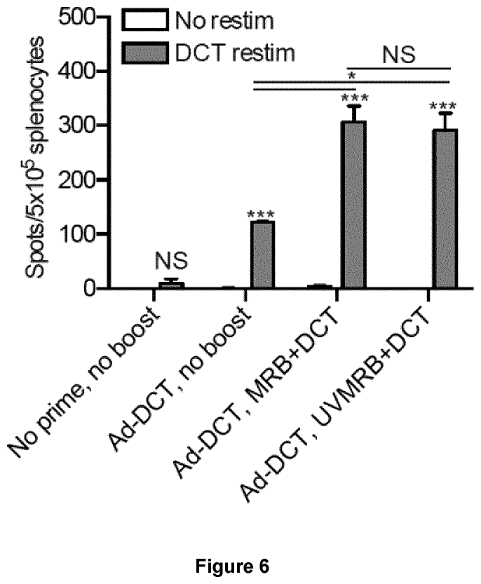

FIG. 6 shows IFNγ ELISPOT analysis of splenocytes harvested on day 21 from mice primed with Ad-DCT and boosted with either MRB or UV-inactivated MRB (UVMRB) co-administered with DCT peptide. The results show that both MRB (third group from the left) and UV-inactivated MRB (UVMRB) (right-most group) provide comparable immune boosting. FIG. 6 thus shows that MRB does not have to replicate (or be oncolytic) in order to boost the antigen-specific immune response in the adjuvant setting. Unless indicated otherwise, the stats refer to the comparison between the “No restim” and “DCT restim” conditions. NS: p>0.05, *: p<0.05, ***: p<0.001 (unpaired multiple two-tailed t-test).

Other oncolytic viruses (OVs) can also be used as adjuvants for immune priming or boosting.

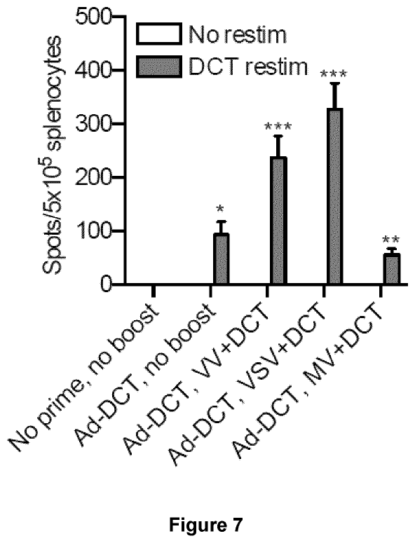

FIG. 7 shows IFNγ ELISPOT analysis of splenocytes harvested on day 21 from mice primed with Ad-DCT and boosted with either VV, VSV or MV co-administered with DCT peptide. The results show once again that Ad-DCT alone induces an immune response to DCT (second group from the left). This immune response could be efficiently boosted by the co-administration of VV (third group from the left) or VSV (fourth group from the left) but not MV (last group) together with DCT peptide. This figure shows that VSV and VV can also be use as adjuvants for immune boosting. The stats refer to the comparison between the “No restim” and “DCT restim” conditions. NS: p>0.05, *: p<0.05, **: p<0.01, ***: p<0.001 (unpaired multiple two-tailed t-test).

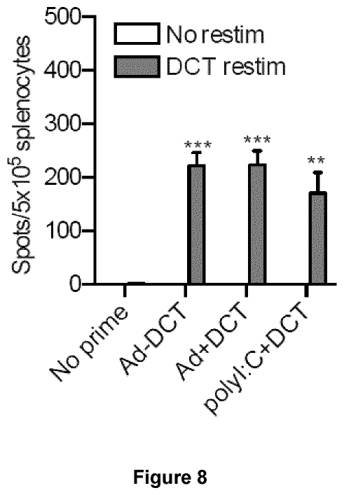

FIG. 8 shows IFNγ ELISPOT analysis of splenocytes harvested on day 14 from mice primed with Ad-DCT or Ad or polyl:C co-administered with DCT peptide (all IM). The results show that the co-administration of the DCT peptide with Ad (third group from the left) or polyl:C (last group) confers comparable priming efficiency to Ad-DCT (second group from the left). This figure shows that Ad can be used as an adjuvant to prime anti-tumour immunity. The stats refer to the comparison between the “No restim” and “DCT restim” conditions. NS: p>0.05, **: p<0.01, ***: p<0.001 (unpaired multiple two-tailed t-test).

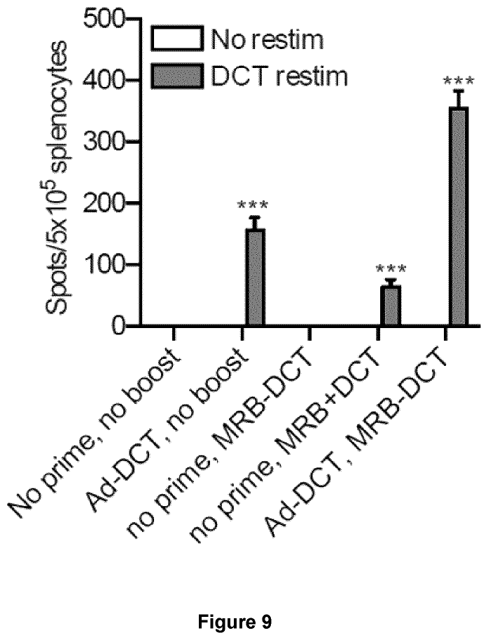

FIG. 9 shows IFNγ ELISPOT analysis of splenocytes harvested on day 21 from mice primed with Ad-DCT and boosted with MRB-DCT or MRB co-administered with DCT peptide. The results show that co-administration of the DCT peptide with MRB (fourth group from the left), but not MRB-DCT (third group from the left) is able to induce a DCT-specific immune response in absence of previous immune priming. This figure shows that MRB can also be used as an adjuvant to prime anti-tumour immunity. The stats refer to the comparison between the “No restim” and “DCT restim” conditions. NS: p>0.05, ***: p<0.001 (unpaired multiple two-tailed t-test).

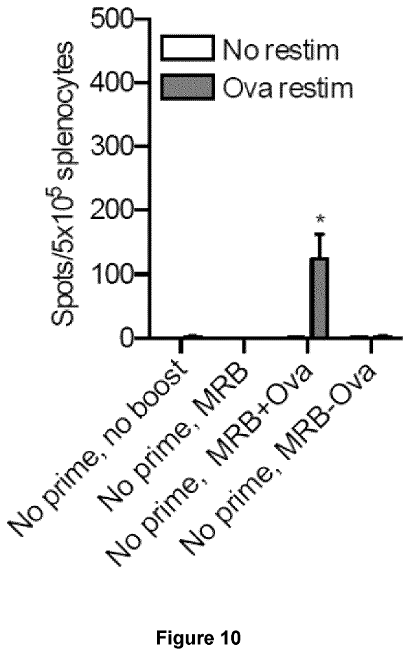

FIG. 10 shows IFNγ ELISPOT analysis of splenocytes harvested on day 14 from mice primed with MRB, MRB-Ova or MRB co-administered with Ova peptide. The results show that only the co-administration of the Ova peptide with MRB (third group from the left) is able to induce Ova-specific immunity in absence of previous immune priming. This figure shows that MRB can also be used as an adjuvant to prime anti-tumour immunity. The stats refer to the comparison between the “No restim” and “Ova restim” conditions. NS: p>0.05, *: p<0.05 (unpaired multiple two-tailed t-test).

MRB can be used as an adjuvant together with mutanome epitopes.

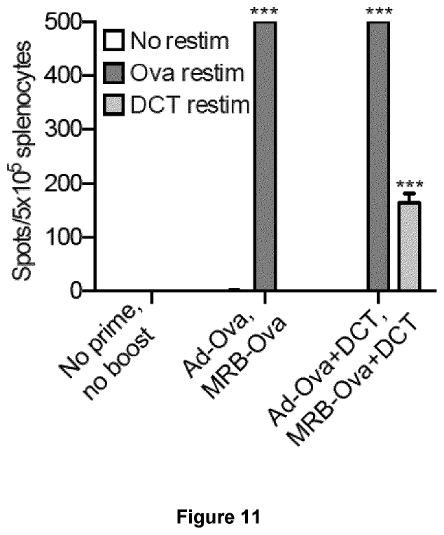

FIG. 11 shows IFNγ ELISPOT analysis of splenocytes harvested on day 21 from mice primed with Ad-Ova together or not with DCT peptide and boosted with MRB-Ova together or not with DCT peptide. The results show that the co-administration of peptide does not impair the immune response induced against the encoded antigen (Ova) (last group vs second group). Also, the co-administration of DCT peptide with both Ad-Ova and MRB-Ova allows for the induction of a DCT immune response (last group), showing that an effective immune response can be generated to a peptide that is not encoded by either the prime virus or the boost virus. FIG. 11 also shows that Ad and MRB platforms encoding antigens can be used together with additional peptides to prime and boost anti-tumour immunity. The stats refer to the comparison between the “No restim” and “peptide restim” conditions. NS: p>0.05, ***: p<0.001 (unpaired multiple two-tailed t-test).

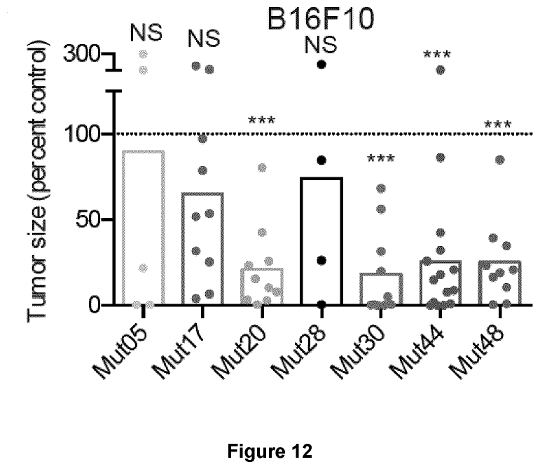

FIG. 12 shows results for mice bearing established subcutaneous B16F10-Ova tumours treated IM with polyl:C and the indicated peptides on days 7 and 14. The tumours were measured on day 21. The tumour volumes are relative to the average tumour volume of control mice (treated with polyl:C only). The results show that B16Mut-20, -30, -44 and 48 have therapeutic activity. NS: p>0.05, ***: p<0.001 (unpaired multiple two-tailed t-test).

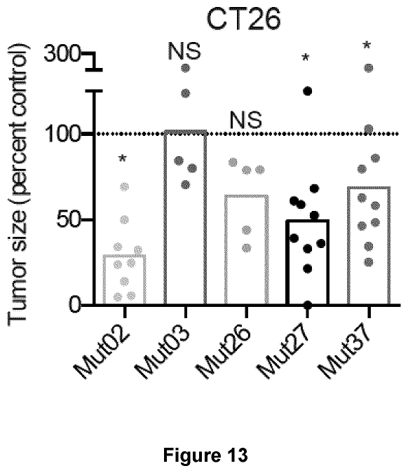

FIG. 13 shows results for mice bearing established subcutaneous CT26 tumours treated IM with polyl:C and the indicated peptide on days 7 and 14. The tumours were measured on day 21. The tumour volumes are relative to the average tumour volume of control mice (treated with polyl:C only). The results show that CT26Mut-02, -27 and -37 have therapeutic activity. NS: p>0.05, *: p<0.05 (unpaired multiple two-tailed t-test).

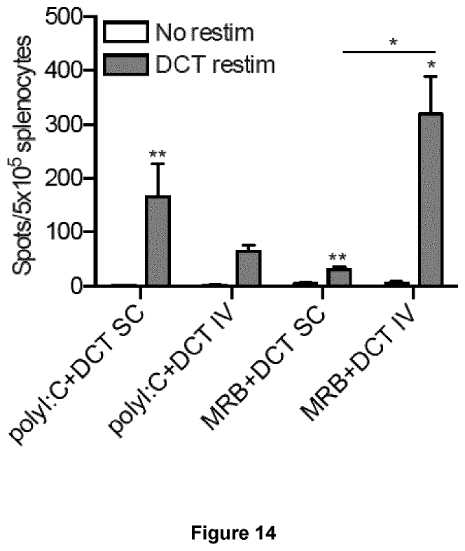

FIG. 14 shows IFNγ ELISPOT analysis of splenocytes harvested on day 14 from mice primed with polyl:C or MRB together with DCT peptide (SC and IV). The results show that all routes and adjuvants could induce a DCT-specific immune response. Importantly, the best routes of administration were SC for polyl:C (first group) and IV for MRB (last group). Notably, there was no statistical difference when comparing the immune priming activity of polyl:C SC (first group) to MRB IV (last group). This FIG. shows that MRB IV is as good as an adjuvant as polyl:C SC. Unless indicated otherwise, the stats refer to the comparison between the “No restim” and “DCT restim” conditions. NS: p>0.05, *: p<0.05, **: p<0.01 (unpaired multiple two-tailed t-test).

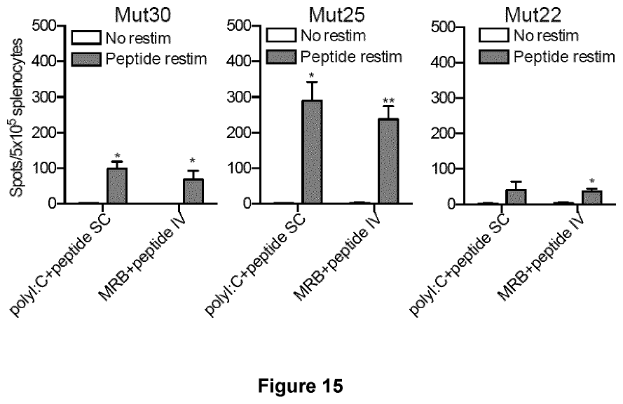

FIG. 15 shows IFNγ ELISPOT analysis of splenocytes harvested on day 14 from mice primed with polyl:C (SC) or MRB (IV) together with the indicated B16Mut peptide. The results show that for all B16Mut peptides tested, MRB IV (second group) is as efficient as polyl:C SC (first group) at inducing a peptide-specific immune response. This figure confirms that MRB IV is as good as an adjuvant as polyl:C SC. The stats refer to the comparison between the “No restim” and “Peptide restim” conditions. NS: p>0.05, *: p<0.05, **: p<0.01 (unpaired multiple two-tailed t-test).

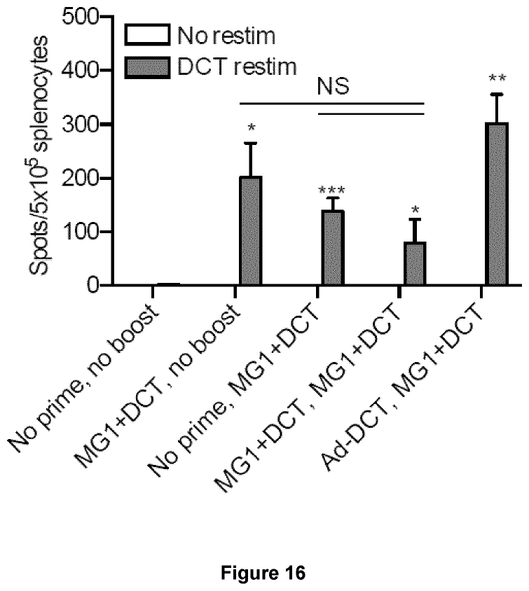

FIG. 16 shows that MRB can be used as an adjuvant for immune priming or boosting, but not both. It depicts the result of IFNγ ELISPOT analysis of splenocytes harvested on day 21 from mice primed with Ad-DCT or MRB together with DCT peptide and boosted with MRB co-administered with DCT peptide. The results show once again that MRB co-administered with DCT peptide can trigger a DCT-specific immune response in absence of previous immune priming (second and third groups from the left). Importantly, repeated administration (days 7 and 14) of MRB together with peptide (fourth group from the left) does not improve the DCT-specific immune response compared to a single administration (second and third groups from the left). This figure shows that a single injection of MRB and peptide is as efficient as multiple injections at inducing antigen-specific immunity. Unless indicated otherwise, the stats refer to the comparison between the “No restim” and “DCT restim” conditions. NS: p>0.05, *: p<0.05, **: p<0.01, ***: p<0.001 (unpaired multiple two-tailed t-test).

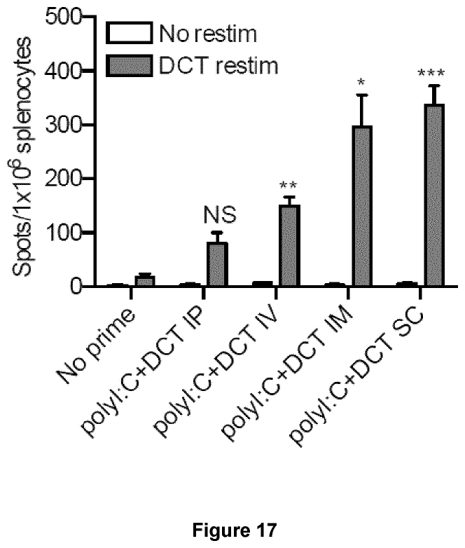

FIG. 17 shows that polyl:C induces stronger immune responses when administered together with peptide IM or SC. It depicts results of IFNγ ELISPOT analysis of splenocytes harvested on day 14 from mice primed with polyl:C co-administered with DCT peptide following different routes (IP, IV, IM or SC). The results show that all routes of administration of polyl:C and peptide induce DCT-specific immunity. Also, the best results were obtained using the IM (fourth group from the left) or SC (last group) routes of administration. This figure shows that the best routes of administration for polyl:C are IM and SC. The stats refer to the comparison between the “No restim” and “DCT restim” conditions. NS: p>0.05, *: p<0.05, **: p<0.01, ***: p<0.001 (unpaired multiple two-tailed t-test).

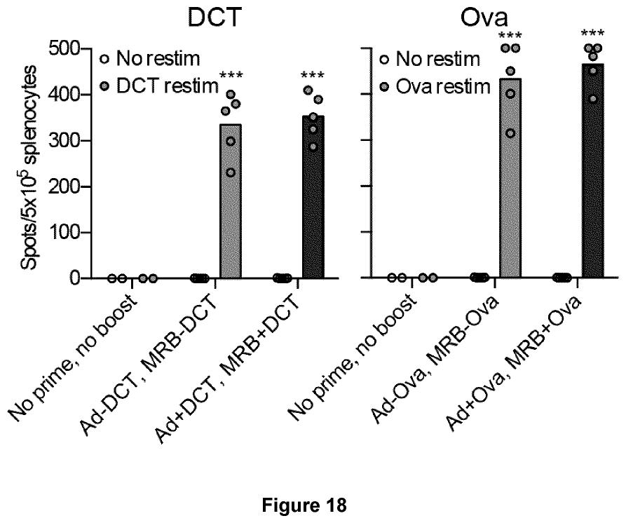

FIG. 18 shows that both Ad and MRB can be used as adjuvants in the heterologous virus prime-boost setting. It depicts the result of IFNγ ELISPOT analysis of splenocytes harvested on day 21 from mice primed with Ad-DCT or Ad together with DCT peptide (day 7) and boosted with MRB-DCT or MRB co-administered with DCT peptide (day 14) (left graph). The right graph is a repeat of the experiment using the Ova model instead of the DCT model. The results show that Ad and MRB co-administered with DCT or Ova peptide can trigger an antigen-specific immune response as efficiently as Ad and MRB encoding DCT or Ova. The stats refer to the comparison between the “No restim” and “restim” conditions. ***: p<0.001 (unpaired multiple two-tailed t-test).

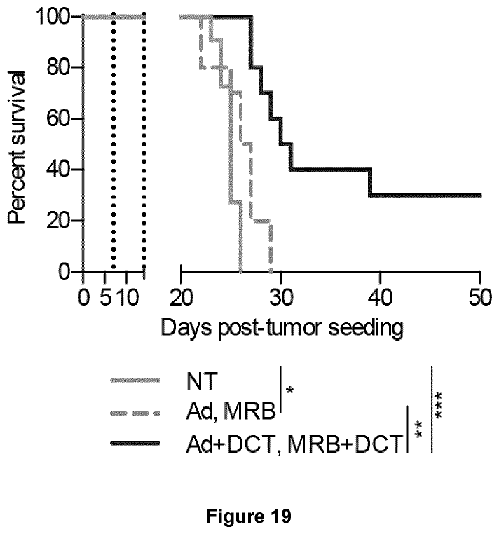

FIG. 19 shows that both Ad and MRB can be used as adjuvants in the heterologous virus prime-boost setting and confer survival benefits. It depicts the survival analysis of mice primed with Ad or Ad together with DCT peptide (day 7) and boosted with MRB or MRB co-administered with DCT peptide (day 14). The results show that Ad and MRB co-administered with DCT peptide can prolong survival of the animals and cure 30% of the mice. Stats: p>0.05, *: p<0.05, **: p<0.01, ***: p<0.001 (Mantel-Cox test).

FIG. 20 shows that both Ad and MRB can be used as adjuvants in the heterologous virus prime-boost setting targeting tumor-specific mutations and confer survival benefits in the B16F10 lung cancer model. It depicts the survival analysis of mice primed with Ad or Ad together with mutanome peptides (B16Mut20, B16Mut30, B16Mut44 and B16Mut48) (day 7) and boosted with MRB or MRB co-administered with mutanome peptides (B16Mut20, B16Mut30, B16Mut44 and B16Mut48) (day 14). The results show that Ad and MRB co-administered with mutanome peptides can prolong survival of the animals and cure 20% of the mice. Stats: NS: p>0.05, ***: p<0.001 (Mantel-Cox test).

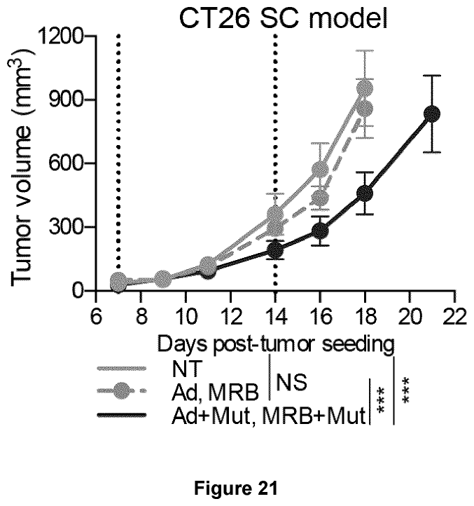

FIG. 21 shows that both Ad and MRB can be used as adjuvants in the heterologous virus prime-boost setting targeting tumor-specific mutations and confer survival benefits in the CT26 SC model. It depicts the tumor growth analysis of mice primed with Ad or Ad together with mutanome peptides (CT26Mut20, CT26Mut27 and CT26Mut37) (day 7) and boosted with MRB or MRB co-administered with mutanome peptides (CT26Mut20, CT26Mut27 and CT26Mut37) (day 14). The results show that Ad and MRB co-administered with mutanome peptides can control the growth of the SC tumors. Stats: NS: p>0.05, ***: p<0.001 (unpaired two-tailed t-test).

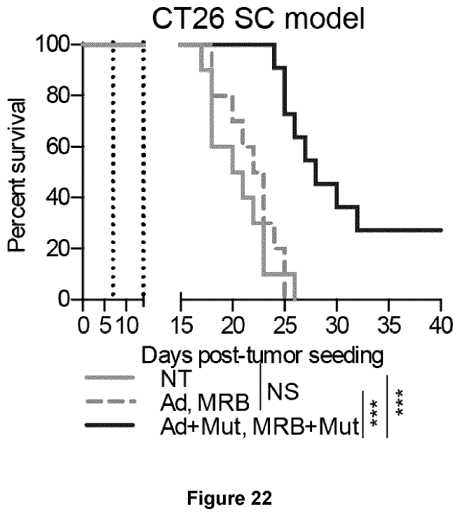

FIG. 22 shows that both Ad and MRB can be used as adjuvants in the heterologous virus prime-boost setting targeting tumor-specific mutations and confer survival benefits in the CT26 SC model. This is the survival analysis from the experiment in FIG. 21. It depicts the survival of mice primed with Ad or Ad together with mutanome peptides (CT26Mut20, CT26Mut27 and CT26Mut37) (day 7) and boosted with MRB or MRB co-administered with mutanome peptides (CT26Mut20, CT26Mut27 and CT26Mut37) (day 14). The results show that Ad and MRB co-administered with mutanome peptides can prolong the survival of the animals and cure a more than 20% of the mice. Stats: NS: p>0.05, ***: p<0.001 (Mantel-Cox test).

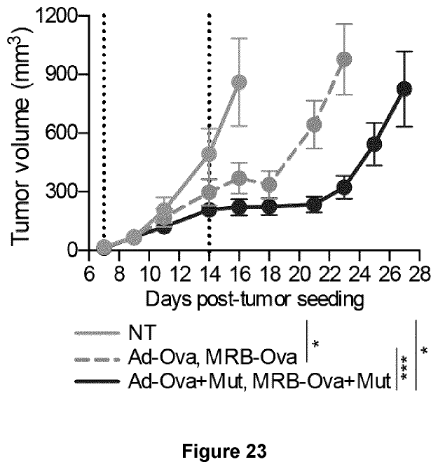

FIG. 23 shows that both Ad and MRB encoding Ova can be used as adjuvants in the heterologous virus prime-boost setting targeting tumor-specific mutations and confer survival benefits in the B16F10-Ova SC model. It depicts the tumor growth analysis of mice primed with Ad-Ova or Ad-Ova together with mutanome peptides (B16Mut20, B16Mut30, B16Mut44 and B16Mut48) (day 7) and boosted with MRB-Ova or MRB-Ova co-administered with mutanome peptides (B16Mut20, B16Mut30, B16Mut44 and B16Mut48) (day 14). The results show that Ad-Ova and MRB-Ova co-administered with mutanome peptides can control the growth of the SC tumors. Stats: *: p<0.05, ***: p<0.001 (unpaired two-tailed t-test).

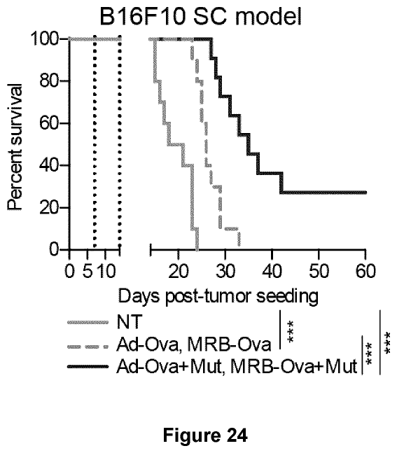

FIG. 24 shows that both Ad-Ova and MRB-Ova can be used as adjuvants in the heterologous virus prime-boost setting targeting tumor-specific mutations and confer survival benefits in the B16F10-Ova SC model. This is the survival analysis from the experiment in FIG. 23. It depicts the survival of mice primed with Ad-Ova or Ad-Ova together with mutanome peptides (B16Mut20, B16Mut30, B16Mut44 and B16Mut48) (day 7) and boosted with MRB-Ova or MRB-Ova co-administered with mutanome peptides (B16Mut20, B16Mut30, B16Mut44 and B16Mut48) (day 14). The results show that Ad-Ova and MRB-Ova co-administered with mutanome peptides can confer survival benefits. Stats: ***: p<0.001 (Mantel-Cox test).

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A method for use in inducing an immune response in a mammalian subject, comprising:
a. administering a prime comprising at least one antigenic protein capable of generating an immune response in the mammal; and
b. administering a boost comprising an oncolytic virus and at least one antigenic protein, formulated to induce the immune response in the mammal;
wherein said at least one antigenic protein of the prime and said at least one antigenic protein of the boost are derived from the same tumour antigen, and
wherein the at least one antigenic protein of the boost is not encoded by the virus of the boost.

2. The method according to claim 1, wherein the amino acid sequence of at least one antigenic protein of the prime and the amino acid sequence of at least one antigenic protein of the boost are at least 70% identical.

3. The method according to claim 2, wherein the amino acid sequence of at least one antigenic protein of the prime and the amino acid sequence of at least one antigenic protein of the boost are at least 80% identical.

4. The method according to claim 3, wherein the amino acid sequence of at least one antigenic protein of the prime and the amino acid sequence of at least one antigenic protein of the boost are at least 90% identical.

5. The method according to claim 4, wherein the amino acid sequence of at least one antigenic protein of the prime and the amino acid sequence of at least one antigenic protein of the boost are identical.

6. The method according to claim 1, wherein:
a. the at least one antigenic protein of the prime comprises a plurality antigenic proteins, and the at least one antigenic protein of the boost comprises a plurality of antigenic proteins, each of which is not encoded by the virus of the boost, and
b. the plurality of antigenic proteins of the prime and the plurality of antigenic proteins of the boost are based on the same plurality of tumour associated antigens.

7. The method according to claim 6, wherein the plurality of antigenic proteins of the prime and the plurality of antigenic proteins of the boost are identical.

8. The method according to any one of claims 1 to 7, wherein the virus of the boost is an oncolytic virus.

9. The method according to claims 1 to 8, wherein the virus of the boost is a Rhabdovirus.

10. The method according to claim 9, wherein the Rhabdovirus is a Maraba virus.

11. The method according to claim 10, wherein the Maraba virus is a MG1.

12. The method according to any one of claims 1 to 7, wherein the virus of the boost is an adenovirus, a vaccinia virus, or a vesicular stomatitis virus.

13. The method according to any one of claims 1 to 12, wherein the boost is formulated for intravenous, intramuscular, or intratumoral administration.

14. The method according to any one of claims 1 to 13, wherein the prime is formulated for intravenous, intramuscular, or intratumoral administration.

15. The method according to any one of claims 1 to 14, wherein the virus of the boost is inactivated.

16. The method according to claim 15, wherein the virus of the boost is UV-inactivated.

17. The method according to any one of claims 1 to 16, wherein the prime additionally comprises a non-viral adjuvant.

18. The method according to any one of claims 1 to 17, wherein the prime additionally comprises a virus, wherein the virus of the prime is immunologically distinct from the virus of the boost.

19. The method according to claim 18, wherein the virus of the prime is an adenovirus.

20. The method according to any one of claims 17 to 19, wherein the virus of the prime is inactivated.

21. The method according to claim 20, wherein the virus of the prime is UV inactivated.

22. A prime:boost vaccine for use in inducing an immune response in a mammalian subject, wherein:
a. said prime comprises at least one antigenic protein capable of generating an immune response in the mammal; and
b. said boost comprises an oncolytic virus and at least one antigenic protein, formulated to induce the immune response in the mammal;
wherein said at least one antigenic protein of the prime and said at least one antigenic protein of the boost are derived from the same tumour antigen, and
wherein the at least one antigenic protein of the boost is not encoded by the virus of the boost.

23. The prime:boost vaccine for use according to claim 22, wherein the amino acid sequence of at least one antigenic protein of the prime and the amino acid sequence of at least one antigenic protein of the boost are at least 70% identical.

24. The prime:boost vaccine for use according to claim 23, wherein the amino acid sequence of at least one antigenic protein of the prime and the amino acid sequence of at least one antigenic protein of the boost are at least 80% identical.

25. The prime:boost vaccine for use according to claim 24, wherein the amino acid sequence of at least one antigenic protein of the prime and the amino acid sequence of at least one antigenic protein of the boost are at least 90% identical.

26. The prime:boost vaccine for use according to claim 25, wherein the amino acid sequence of at least one antigenic protein of the prime and the amino acid sequence of at least one antigenic protein of the boost are identical.

27. The prime:boost vaccine for use according to claim 22, wherein:
a. the at least one antigenic protein of the prime comprises a plurality antigenic proteins, and the at least one antigenic protein of the boost comprises a plurality of antigenic proteins, each of which is not encoded by the virus of the boost, and
b. the plurality of antigenic proteins of the prime and the plurality of antigenic proteins of the boost are based on the same plurality of tumour associated antigens.

28. The prime:boost vaccine for use according to claim 27, wherein the plurality of antigenic proteins of the prime and the plurality of antigenic proteins of the boost are identical.

29. The prime:boost vaccine for use according to any one of claims 22 to 28, wherein the virus of the boost is an oncolytic virus.

30. The prime:boost vaccine for use according to claim 29, wherein the virus of the boost is a Rhabdovirus.

31. The prime:boost vaccine for use according to claim 30, wherein the Rhabdovirus is a Maraba virus.

32. The prime:boost vaccine for use according to claim 31, wherein the Maraba virus is a MG1.

33. The prime:boost vaccine for use according to any one of claims 22 to 28, wherein the virus of the boost is an adenovirus, a vaccinia virus, or a vesicular stomatitis virus.

34. The prime:boost vaccine for use according to any one of claims 22 to 33, wherein the boost is formulated for intravenous, intramuscular, or intratumoral administration.

35. The prime:boost vaccine for use according to any one of claims 22 to 34, wherein the prime is formulated for intravenous, intramuscular, or intratumoral administration.

36. The prime:boost vaccine for use according to any one of claims 22 to 34, wherein the virus of the boost is inactivated.

37. The prime:boost vaccine for use according to claim 36, wherein the virus of the boost is UV-inactivated.

38. The prime:boost vaccine for use according to any one of claims 22 to 37, wherein the prime additionally comprises a non-viral adjuvant.

39. The prime:boost vaccine for use according to any one of claims 22 to 38, wherein the prime additionally comprises a virus, wherein the virus of the prime is immunologically distinct from the virus of the boost.

40. The prime:boost vaccine for use according to anyone of claims 22 to 39, wherein the virus of the prime is an adenovirus.

41. A kit for use in inducing an immune response in a mammalian subject, wherein the kit comprises:
a. a prime that comprises at least one antigenic protein capable of generating an immune response in the mammal; and
b. a boost that comprises an oncolytic virus and at least one antigenic protein, formulated to induce the immune response in the mammal;
wherein said at least one antigenic protein of the prime and said at least one antigenic protein of the boost are derived from the same tumour antigen, and
wherein the at least one antigenic protein of the boost is not encoded by the virus of the boost.

42. The kit according to claim 41, wherein the at least one antigenic protein of the prime and the at least one antigenic protein of the boost are the same.

43. The kit according to claim 41, wherein:
a. the at least one antigenic protein of the prime comprises a plurality antigenic proteins, and the at least one antigenic protein of the boost comprises a plurality of antigenic proteins, each of which is not encoded by the virus of the boost,
b. wherein the plurality of antigenic proteins of the prime and the plurality of antigenic proteins of the boost are based on the same plurality of tumour associated antigens.

44. The kit according to claim 43, wherein the plurality of antigenic proteins of the prime and the plurality of antigenic proteins of the boost are the same.

45. The kit according to any one of claims 41 to 44, wherein the virus of the boost is an oncolytic virus.

46. The kit according to claim 45, wherein the oncolytic virus is a Rhabdovirus.

47. The kit according to claim 46, wherein the Rhabdovirus is a Maraba virus or an engineered variant thereof.

48. The kit according to claim 47, wherein the Maraba virus is MG1.

49. The kit according to any one of claims 41 to 44, wherein the virus of the boost is an adenovirus, a vaccinia virus, or a vesicular stomatitis virus.

50. The kit according to any one of claims 41 to 49, wherein the boost is formulated for intravenous, intramuscular, or intratumoral administration.

51. The kit according to any one of claims 41 to 50, wherein the prime is formulated for intravenous, intramuscular, or intratumoral administration.

52. The kit according to any one of claims 41 to 51, wherein the virus of the boost is inactivated.

53. The kit according to claim 52, wherein the virus of the boost is UV-inactivated.

54. The kit according to any one of claims 41 to 53, wherein the prime additionally comprises a non-viral adjuvant.

55. The kit according to any one of claims 41 to 54, wherein the prime additionally comprises a virus, wherein the virus of the prime is immunologically distinct from the virus of the boost.

56. The kit according to claim 55, wherein the virus of the prime is an adenovirus.

57. The kit according to any one of claims 41 to 56, wherein the at least one antigenic protein of the prime is/are not encoded by the virus of the prime.

58. The kit according to any one of claims 41 to 57, wherein the virus of the prime is inactivated.

59. The kit according to claim 58, wherein the virus of the prime is UV inactivated.
